(19) United States
(12) Patent Application Publication
(10) Pub. No.: US 2025/0121173 A1
(43) Pub. Date: Apr. 17, 2025

(54) DEVICE AND METHOD FOR REDUCING BLOCKAGE IN TUBING SYSTEMS

(71) Applicant: Cottonwood Medical, LLC, Mission Hills, KS (US)

(72) Inventor: Michael Meurer, Mission Hills, KS (US)

(21) Appl. No.: 18/488,025

(22) Filed: Oct. 16, 2023

(51) Int. Cl. A61M 39/10; A61M 25/00; A61M 27/00; A61M 39/20

(52) U.S. Cl. CPC A61M 39/105; A61M 27/00; A61M 39/20; A61M 2025/0019

(57) ABSTRACT

The present invention relates to apparatus, systems, methods, and uses relating to drainage or small bore tubing systems that are primarily used to remove waste fluids or gases during postoperative care after surgery. The systems and methods of the present invention are particularly applicable for use in patients who have undergone cardiac or other thoracic surgeries.

### BACKGROUND OF THE INVENTION

### A. Field of the Invention

The present invention relates generally to apparatus, systems, methods, and uses relating to drainage or small bore tubing systems for medical or veterinary purposes. In particular, the invention relates to systems and methods used for postoperatively draining fluids and/or gases from patients.

### B. Description of the Related Art

In the practice of medicine, post-operative complications are detrimental to patient outcomes. Such complications can be especially deleterious after cardiac or other thoracic surgeries and may require further surgical intervention to resolve. During the hours post-surgery when a patient is still bleeding, it is vitally important that the bodily fluids associated with such bleeding can be drained away so that the patient may recover. Draining away such bodily fluids, as well as any extraneous gases, from a postoperative patient can be difficult due to the natural coagulative properties of blood.

Blood clots can begin to block conventional chest tubes and drainage systems almost immediately after they are inserted. Over time these blockages often grow and can completely block a drainage system unless they are removed. Conventional techniques such as milking, stripping, wiggling, or the use of suction to clear a drainage system or tubing can be unproductive and potentially harmful to a patient.

The drainage apparatus for cardiopulmonary bypass circuits or post-surgical chest drainage systems frequently include one or more Y connectors that are external to the patient and attached to tubing(s) that enter into the chest cavity and through which bodily waste is to be drained from the patient. A “Y” connector is a device that is used to direct the flow of fluids or gases from two separate channels into a single channel.

Currently available Y connectors exist both with and without additional ports. These additional ports are typically oriented in a perpendicular fashion to the flow of fluids or gases passing through the Y connector. These additional ports are typically used for sampling or adding fluid or gaseous substances into the system channels. Due to their perpendicular orientation to the system channels, these additional ports are not adapted for the insertion and advancement of a tool, instrument, or suction catheter into the system channels to remove obstructions, debris, or clots that block the drainage of fluids or gases from the patient. Consequently, to remove a blockage in the drainage system, it is not uncommon for a user to have to disconnect the Y connector from the tubing so that a suitable tool can be inserted into the tubing to remove the blockage. Unfortunately, disconnecting the Y connector can result in the loss of a sterile field for the drainage system, which increases the risk of infection to the patient. To avoid the loss of the sterile field, either multiple users are needed to handle the drainage system or a user must replace at least the used Y connector with a new sterile one. Because blockage can recur multiple times, the expense and risk to a patient can increase postoperatively.

### SUMMARY OF THE INVENTION

The present invention provides drainage systems that include a sterile field and methods to repeatedly manipulating the systems while maintaining a sterile field without requiring either the replacement of parts in the systems or a second user. The invention is expected to be particularly useful in drainage systems that are used for the postoperative care of thoracic surgery patients. For example, the invention provides systems and methods that can be used after cardiopulmonary bypass surgery to drain accumulated fluids or gases from the thoracic cavity.

In some embodiments the invention comprises a modified Y connector apparatus that includes a primary inlet channel; a secondary inlet channel; a convergence channel into which fluids, gases, or mixtures of fluids and gases from the primary inlet channel or secondary inlet channel can flow; a first port that is oriented in line with the primary inlet channel; and a second port that is oriented in line with the secondary inlet channel. This Y connector apparatus also includes a first cap configured to seal the first port and a second cap configured to seal the second port.

Advantageously, a user can remove a cap from a port to access the associated inlet channel while leaving the other cap in place over the other respective port. In this manner, a sterile field may be maintained because no portion of the Y connector apparatus is removed from the sterile field. Thus, a second user is not needed to prevent a portion of the Y connector apparatus from coming into contact with a non-sterile surface. Nor is there a need to replace the Y connector apparatus, or a portion of it, after a cap is removed from a port to access the port and its associated inlet channel because the sterile field can be maintained when a user attempts to remove a blockage from the system. Those of skill in the art will appreciate that the sampling or adding of fluid or gaseous substances to the system can also be done by accessing the system in the same manner. And, again, the sterile field can be maintained without the need for a second user or the replacement of a Y connector.

The first cap and the second cap of the Y connector apparatus each include an extension (a gripping extension) such that a user can grasp and remove the cap from its associated port. An extension can be a handle, grip, knob, shaft, or hilt and have a variety of shapes, as long as, a user can grasp the extension to remove a cap from a port and then replace the cap over the port. Preferably, the extension allows a user to remove a cap from a port with one hand.

Preferably, the first cap and second cap are tethered together. The tether is of sufficient length so that one cap can be removed from a port; remain attached to the other cap; and replaced over its associated port. In preferred embodiments, the caps fit snuggly over the ends of the associated ports. In other embodiments, the caps may twist or snap into place over their associated ports. In these embodiments, the tether is of sufficient length to accommodate the additional movement so that the caps remain attached to one another during use.

In certain preferred embodiments, the primary inlet channel, secondary inlet channel, and convergence channel each have exterior ridges, multi-indentations, or a combination thereof sufficiently near, adjacent or proximal to their peripheral openings so that when tubing is attached to a respective channel the tubing is securely attached over the peripheral opening and gravitational force alone does not cause the tubing to slip off of the connector.

Embodiments of the invention comprise a modified Y connector apparatus that has its primary inlet channel, secondary inlet channel, convergence channel, first port, and second port all in fluid communication with one another. Other embodiments of the invention comprise a modified Y connector apparatus that has its single inlet channel, convergence channel, first port, and second port all in fluid communication with one another.

Embodiments of the invention comprise a modified Y connector apparatus composed of a rigid material. Suitable rigid materials are those that can be sterilized and molded or shaped into the Y connector apparatus. For example, metal, plastic, or a combination thereof, are suitable rigid materials. A preferred rigid material is plastic.

The first cap and the second cap can be composed of a rigid material, a flexible material, or a combination thereof. The material used to construct the first and second caps must be able to be sterilized.

Alternative embodiments of the invention comprise a single inlet channel; a convergence channel into which fluids or gases from the single inlet channel may flow; a first port that deviates from being in line with the single inlet channel by an angle equal to or less than 45 degrees; and a second port that deviates from being in line with the single inlet channel by an angle equal to or less than 45 degrees.

The invention further comprises a method of clearing a postoperative drainage system that comprises attaching to the postoperative drainage system a Y connector apparatus having a primary inlet channel; a secondary inlet channel; a convergence channel; a first port that is oriented in line with the primary inlet channel; and a second port that is oriented in line with the secondary inlet channel, wherein the primary inlet channel is attached to a first drainage tube and the secondary inlet channel is attached to a second drainage tube and the convergence channel is attached to a third drainage tube such that fluid or gas can flow from the first and second drainage tubes through the primary and secondary inlet channels into the convergence channel and into the third drainage tube; clearing a blockage in the first drainage tube by inserting a tool, instrument, or suction catheter through the first port into the primary inlet channel and into the first drainage tubing to dislodge the blockage and then removing the tool, instrument, or suction catheter from the first drainage tubing through the primary inlet channel and the first port; and clearing a blockage in the second drainage tube by inserting a tool, instrument, or suction catheter through the second port into the secondary inlet channel and into the second drainage tubing to dislodge the blockage and then removing the tool, instrument, or suction catheter from the second drainage tubing through the secondary inlet channel and the second port.

Another method of the invention for facilitating the clearance a postoperative drainage system comprises attaching to the postoperative drainage system a Y connector apparatus having a single inlet channel; a convergence channel; a first port that is attached to the single inlet channel and deviates from being in line with the single inlet channel by an angle equal to or less than 45 degrees; and a second port that is attached to the single inlet channel and deviates from being in line with the single inlet channel by an angle equal to or less than 45 degrees, wherein the single inlet channel is attached to a drainage tube and the convergence channel is attached to a clearing tube such that fluid or gas can flow from the drainage system through the single inlet channel into the convergence channel and into the clearing tube; and clearing a blockage in the drainage system by inserting a tool, instrument, or suction catheter through the first port into the single inlet channel and into the drainage system to dislodge the blockage and then removing the tool, instrument, or suction catheter from the drainage system through the single inlet channel and the first port, or clearing the blockage in the drainage system by inserting a tool, instrument, or suction catheter through the second port into the single inlet channel and into the drainage system to dislodge the blockage and then removing the tool, instrument, or suction catheter from the drainage system through the single inlet channel and the second port.

Methods of the invention further comprise removing a first cap that seals the first port before inserting the tool, instrument, or suction catheter through the first port and replacing the first cap to seal the first port after removal of the tool, instrument, or suction catheter; and removing a second cap that seals the second port before inserting the tool, instrument, or suction catheter through the second port and replacing the second cap to seal the second port after removal of the tool, instrument, or suction catheter.

Alternatively methods of the invention further comprise removing either a first cap that seals a first port or removing a second cap that seals a second port before inserting a tool, instrument, or suction catheter through the port into its associated inlet channel and drainage tubing, and then after the removal the tool, instrument, or suction catheter replacing either the first cap or the second cap to seal its associated port.

Methods of the invention further comprise that, when one cap is removed from its respective port, the other cap remains attached to the Y connector apparatus and a sterile field is maintained.

Those of skill in the art will recognize that the compositions and methods of the invention may be applicable to other situations in which a drainage or access system may be repeatedly manipulated and it is desirable to maintain a sterile environment.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description. Further, while specific advantages of the invention are detailed herein, various embodiments may include some, none, or all of these enumerated advantages.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description presented herein. Unless specifically noted, articles depicted in the drawings are not necessarily drawn to scale.

FIG. 1A is a front view of a modified Y connector with caps attached over the ports.

FIG. 1B is a bottom view that shows the caps covering the ports and open end of the convergence channel. FIG. 1C is a side view. FIG. 1D is a cross-section as indicated by the double-headed arrow in FIG. 1C with one cap removed from covering a port. FIG. 1E is side perspective view. FIG. 1F is a vertical perspective view.

FIG. 2A is a front view of a modified Y connector without the caps attached. FIG. 2B a top view that shows the open ends of the primary and secondary inlet channels. FIG. 2C is bottom view that shows the ports without caps. FIG. 2D is a side perspective view as indicated by the double-headed arrow in FIG. 2A. FIG. 2E is a side perspective of the modified Y connector without the caps attached. FIG. 2F is a cross-section as indicated by the double-headed arrow in FIG. 2E. FIG. 2G is a vertical side perspective without caps. FIG. 2H is a vertical side perspective without caps and with the convergence channel on top.

FIG. 3A is a top view of the tethered caps. FIG. 3B is a front view of the tethered caps. FIG. 3C is a cross-section of the tethered caps as indicated by the double headed arrow in FIG. 3B. FIG. 3D is a front side perspective of the tethered caps. FIG. 3E is a back side perspective of the tethered caps. FIG. 3F is a back view of the tethered caps. FIG. 3G. is a side view.

FIG. 4A is a front perspective view of another embodiment without the tethered caps in place. The back perspective view is identical (i.e. a mirror image) and not shown. FIG. 4B is a front perspective view of the same embodiment with the tethered caps shown. The back perspective view is identical (i.e. a mirror image) and not shown.

### DETAILED DESCRIPTION

The present invention provides compositions and methods for maintaining a sterile field. The invention is expected to be particularly useful as a chest drainage system in patients having had cardiopulmonary bypass surgery. Temporary drainage systems are necessary in such patients post-operatively, but these systems are often subject to blockage and require manipulation to remove to blood clots or other material that prevents proper drainage.

A conventional Y connector is characterized by a housing having a convergence channel that is a generally tubular central body member having two inlet channels connected to it. The two inlet channels are also generally tubular. The channels may be of the similar lengths or disparate lengths as compared to each other. Further the thicknesses of the channels can be varied as can their interior diameters. Herein is disclosed a modified Y connector that includes additional tubular members. Those of skill in the art will appreciate that the lengths, diameters, and thicknesses of the different parts of the modified Y connector, as well as the associated tethered caps, can be varied without departing from the scope of the disclosed invention. It is only necessary that each part be capable of functioning as described herein.

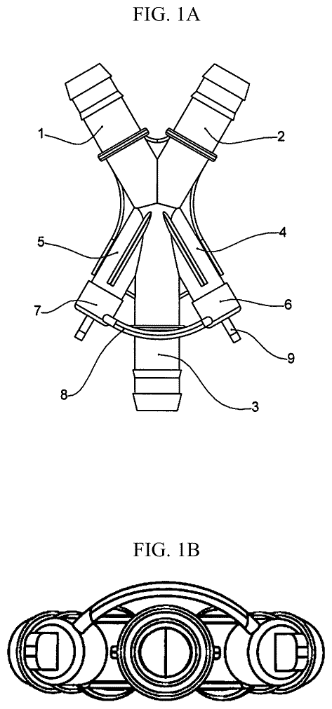

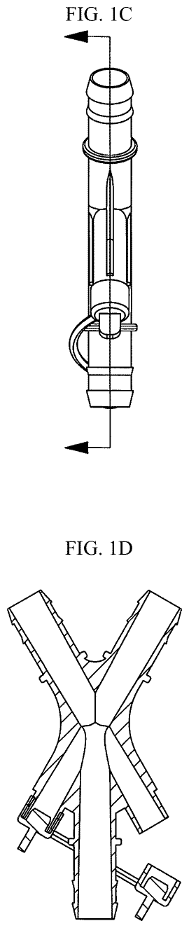

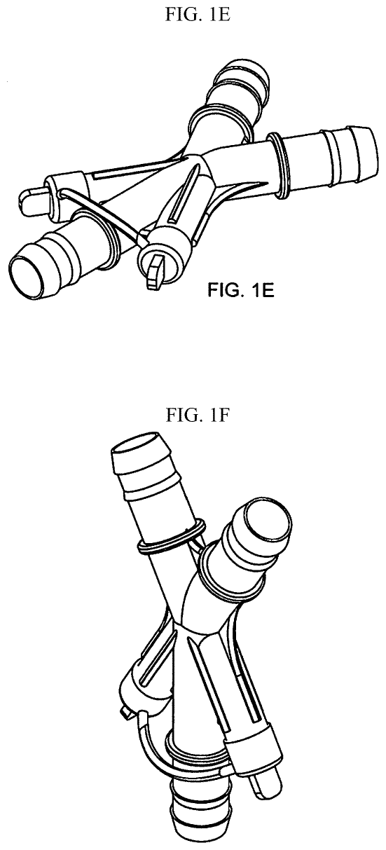

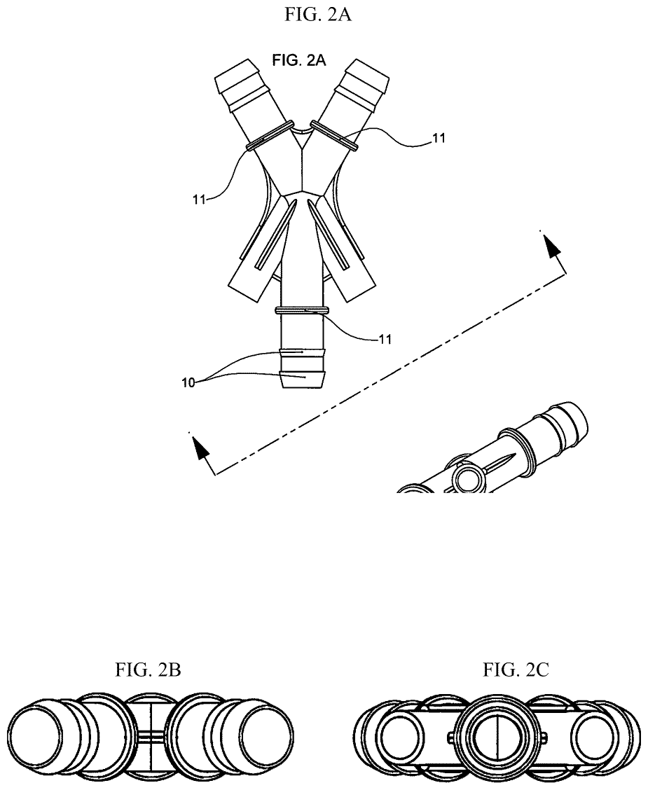

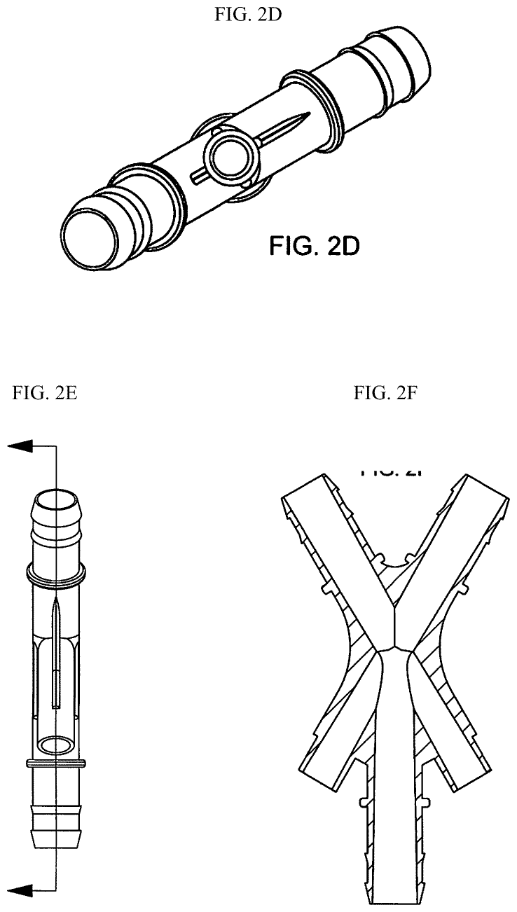

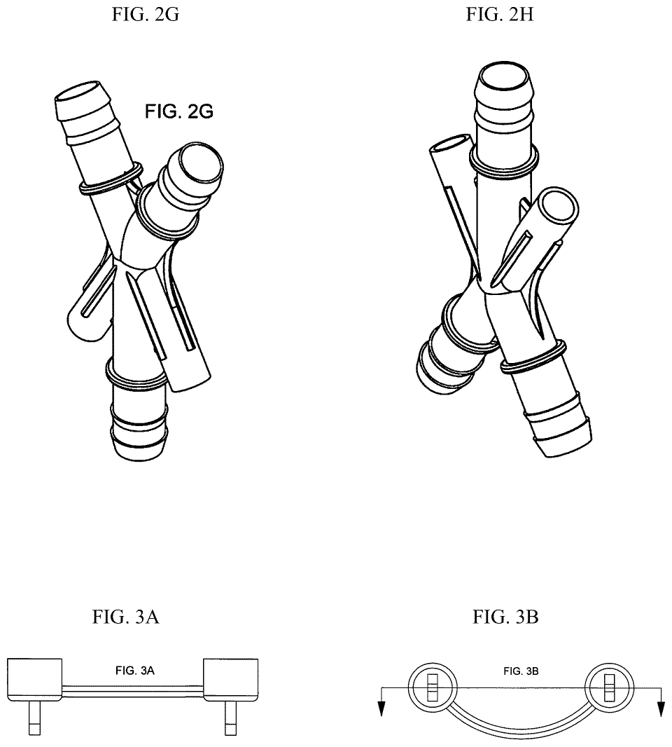

The modified Y connector of the invention comprises a convergence channel 3 that is connected to a primary inlet channel 1 and a secondary inlet channel 2. See FIGS. 1A, 1D, 1E, 1F, 2A, 2F, 2G, and 2H. The modified Y connector also includes two additional tubular members, a first port 4 and a second port 5. The first port 4 is oriented in line with the primary inlet channel 1 such that a tool, instrument, or suction catheter may be inserted into and through the first port 4 so that it enters into the primary inlet channel 1 without the need to bend around a corner or angle, i.e. an essentially straight line may be drawn between the interior of the first port 4 and the interior of the primary inlet channel 1. Similarly, the second port 5 is oriented in line with the secondary inlet channel 2 such that a tool, instrument, or suction catheter may be inserted into and through the second port 5 so that it enters into the secondary inlet channel 2 in an essentially straight line.

In an alternative embodiment, only primary inlet channel 1 is present without a secondary inlet channel, i.e. a single inlet channel is present. In this embodiment, preferably the primary inlet channel 1 is in a direct line with the convergence channel 3. In this embodiment, both the tubular first port 4 and the second port 5 are oriented with the single inlet channel such that a tool, instrument, or suction catheter may be inserted into and through either port 4 or 5 without the need to bend around a significant angle or corner. It will be understood that in this embodiment a tool, instrument, or suction catheter may need to be navigated through an angle into the primary inlet channel 1 (i.e. single inlet channel). Preferably, the angle through with the tool, instrument, or suction catheter is navigated is less than 90 degrees, more preferably the angle is less than 45 degrees, even more preferably the angle is less than 30 degrees, and most preferably the angle is equal to or less than 20 degrees.

Preferably, when not in use for the introduction of a tool or other instrument, the first and second ports are covered so that a sterile field can be maintained. In particular, the first port 4 is covered by a first cap 6, and the second port 5 is covered by a second cap 7. The first cap 6 and second cap 7 are attached to each other by a tether 8. See FIGS. 1A, 1B, 1E, 1F, 3A, 3B, 3C, 3D, 3E, 3F, and 4B. The caps and tether may comprise any suitable material that can be sterilized. The length and thickness of the tether can be varied as long as the tether is of sufficient length and flexibility that one cap may be removed and then replaced repeatedly while the other cap remains in place over its respective port.

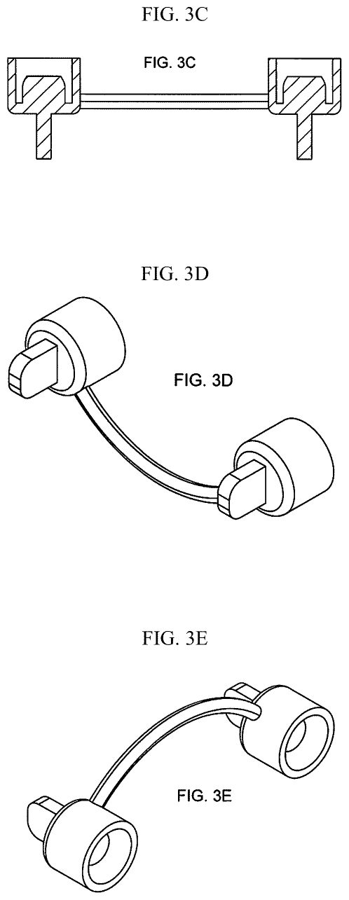

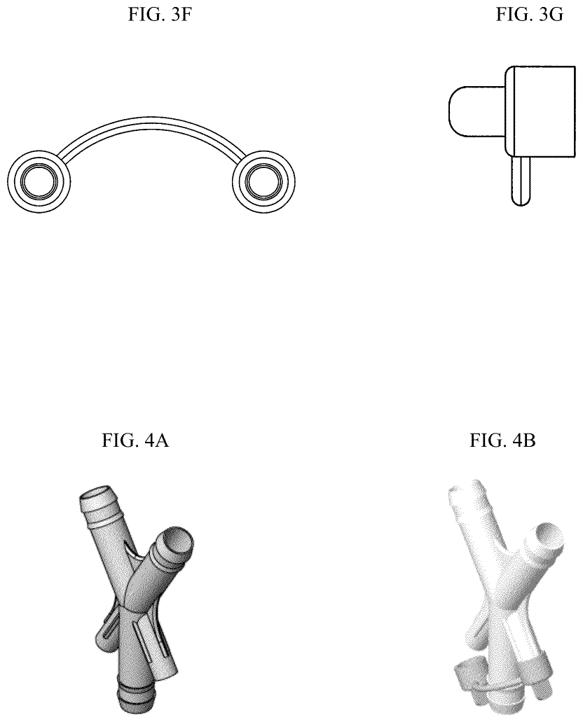

So that a user may more easily attach or remove a cap from a port, each cap includes an extension 9. See FIGS. 3C, 3D, 3E, and 3G. Those of skill in the art will appreciate that the shape and size of the extension can be varied without losing function. In an alternative embodiment, not shown, caps may not include an extension.

Preferred caps cover the open ends of their respective ports and extend slightly into the open ends of those ports. See FIGS. 1C, 1D and 3C. Those of skill in the art will recognize that to function as described, the caps do not necessarily need to extend into the ports.

Preferably, the exterior surfaces of the primary inlet channel 1, secondary inlet channel 2, and convergence channel 3 each include exterior ridges 10 or multi-indentations 10 or a combination thereof. See FIGS. 1A and 2A. The exterior ridges, multi-indentations, or combinations thereof are located near or adjacent to the respective open end of each channel.

Those of skill in the art will appreciate that the configuration of such exterior ridges of multi-indentations can be varied. It is only necessary that a tubing can be placed over the exterior ridges, multi-indentations, or combinations thereof such that the tubing is attached to the modified Y connector and the tubing cannot be displaced from the modified Y connector solely by gravitational force or removed without the application of some external force. For example, the tubing cannot be bumped and accidentally separated from the modified Y connector.

In addition, it is preferred that the exterior surfaces of the primary inlet channel 1, secondary inlet channel 2, and convergence channel 3 each include a tube stop 11. See FIG. 2A. Skilled artisans will recognize that the configuration of the tube stop can be varied without losing function. The tube stop functions to prevent tubing from being attached to the modified Y connector such that either one tubing is in contact with another tubing that is connected to a different channel or that a user cannot easily remove a cap from a port without endangering the loss of the sterile field. Alternative embodiments of the invention do not necessarily include tube stops. See FIGS. 4A and 4B.

Unless defined otherwise, all technical and scientific terms used herein have the same meaning as is commonly understood by one of skill in the art to which this invention belongs at the time of filing. Terminology used herein is for the purpose of describing particular embodiments of the invention and is not intended to be limiting. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular unless the content clearly dictates otherwise. Herein, the use of “or” means “and/or” unless stated otherwise. Furthermore, the use of the term “including”, as well as other forms such as “includes” and “included” is not limiting. Similarly, the use of the term “comprising” is not limiting. As used herein, “each” refers to each member of a set or each member of a subset of a set.

It should be understood that, although exemplary embodiments are illustrated and described herein, the principles of the present disclosure may be implemented using a variety of techniques, whether or not currently known in the art. Thus, the present disclosure should not be limited to exemplary embodiments and techniques illustrated herein. Modifications, additions, or omissions may be made to the systems, apparatuses, and methods described herein without departing from the scope of the disclosure. For example, unless otherwise indicated the components of the systems and apparatuses may be integrated or separated. Moreover, the operations of the systems and apparatuses disclosed herein may be performed by more, fewer, or other components and the methods described may include more, fewer, or other steps. Additionally, unless otherwise indicated steps may be performed in any suitable order.

What is claimed is:

1. A Y connector apparatus comprising
a) a primary inlet channel;
b) a secondary inlet channel;
c) a convergence channel into which fluids or gases from the primary inlet channel and secondary inlet channel may flow;
d) a first port that is oriented in line with the primary inlet channel; and
e) a second port that is oriented in line with the secondary inlet channel.

2. The Y connector apparatus of claim 1 further comprising a first cap configured to seal the first port and a second cap configured to seal the second port, wherein the first cap and second cap are tethered together.

3. The Y connector apparatus of claim 2, wherein the first cap and the second cap are composed of a rigid material, a flexible material, or a combination thereof.

4. The Y connector apparatus of claim 2, wherein the first cap and the second cap each include a gripping extension.

5. The Y connector apparatus of claim 1, wherein the primary inlet channel, secondary inlet channel, and convergence channel have exterior ridges, multi-indentations, or a combination thereof at their peripheral openings.

6. The Y connector apparatus of claim 1, wherein the primary inlet channel, secondary inlet channel, convergence channel, first port, and second port are in fluid communication with one another.

7. The Y connector apparatus of claim 1, wherein the primary inlet channel, secondary inlet channel, convergence channel, first port, and second port are composed of a rigid material.

8. The Y connector apparatus of claim 1, wherein the primary inlet channel, secondary inlet channel, and convergence channel include a tube stop.

9. A method of facilitating a postoperative drainage system comprising
a) attaching to the postoperative drainage system a Y connector apparatus having a primary inlet channel; a secondary inlet channel; a convergence channel; a first port that is oriented in line with the primary inlet channel; and a second port that is oriented in line with the secondary inlet channel, wherein the primary inlet channel is attached to a first drainage tube and the secondary inlet channel is attached to a second drainage tube and the convergence channel is attached to a clearing tube such that fluid or gas can flow from the first and second drainage tubes through the primary and secondary inlet channels into the convergence channel and into the clearing tube;
b) clearing a blockage in the first drainage tube by inserting a tool, instrument, or suction catheter through the first port into the primary inlet channel and into the first drainage tubing to dislodge the blockage and then removing the tool, instrument, or suction catheter from the first drainage tubing through the primary inlet channel and the first port, or clearing a blockage in the second drainage tube by inserting a tool, instrument, or suction catheter through the second port into the secondary inlet channel and into the second drainage tubing to dislodge the blockage and then removing the tool, instrument, or suction catheter from the second drainage tubing through the secondary inlet channel and the second port.

10. The method of claim 9 further comprising removing a first cap that seals the first port before inserting the tool, instrument, or suction catheter through the first port and replacing the first cap to seal the first port after removal of the tool, instrument, or suction catheter; or removing a second cap that seals the second port before inserting the tool, instrument, or suction catheter through the second port and replacing the second cap to seal the second port after removal of the tool, instrument, or suction catheter.

11. The method of claim 10, wherein the first cap and second cap are tethered together such that when one cap is removed the other cap remains attached to the Y connector apparatus, and a sterile field is maintained.

12. A Y connector apparatus comprising
a) a single inlet channel;
b) a convergence channel into which fluids or gases from the single inlet channel may flow;
c) a first port that deviates from being in line with the single inlet channel by an angle equal to or less than 45 degrees; and
d) a second port that deviates from being in line with the single inlet channel by an angle equal to or less than 45 degrees.

13. The Y connector apparatus of claim 12 further comprising a first cap configured to seal the first port and a second cap configured to seal the second port, wherein the first cap and second cap are tethered together.

14. The Y connector apparatus of claim 13, wherein the first cap and the second cap are composed of a rigid material, a flexible material, or a combination thereof.

15. The Y connector apparatus of claim 13, wherein the first cap and the second cap each include an extension.

16. The Y connector apparatus of claim 12, wherein the single inlet channel and convergence channel have exterior ridges, multi-indentations, or a combination thereof at or near their peripheral openings.

17. The Y connector apparatus of claim 12, wherein the single inlet channel, convergence channel, first port, and second port are in fluid communication with one another.

18. The Y connector apparatus of claim 12, wherein the single inlet channel, convergence channel, first port, and second port are composed of a rigid material.

19. The Y connector apparatus of claim 12, wherein the single inlet channel and convergence channel include a tube stop.

20. A method of facilitating a postoperative drainage system comprising
a) attaching to the postoperative drainage system a Y connector apparatus having a single inlet channel; a convergence channel; a first port that is attached to the single inlet channel and deviates from being in line with the single inlet channel by an angle equal to or less than 45 degrees; and a second port that is attached to the single inlet channel and deviates from being in line with the single inlet channel by an angle equal to or less than 45 degrees, wherein the single inlet channel is attached to a drainage tube and the convergence channel is attached to a clearing tube such that fluid or gas can flow from the drainage system through the single inlet channel into the convergence channel and into the clearing tube; and
b) clearing a blockage in the drainage system by inserting a tool, instrument, or suction catheter through the first port into the single inlet channel and into the drainage system to dislodge the blockage and then removing the tool, instrument, or suction catheter from the drainage system through the single inlet channel and the first port, or clearing the blockage in the drainage system by inserting a tool, instrument, or suction catheter through the second port into the single inlet channel and into the drainage system to dislodge the blockage and then removing the tool, instrument, or suction catheter from the drainage system through the single inlet channel and the second port.

21. The method of claim 20 further comprising removing a first cap that seals the first port before inserting the tool, instrument, or suction catheter through the first port and replacing the first cap to seal the first port after removal of the tool, instrument, or suction catheter; or removing a second cap that seals the second port before inserting the tool, instrument, or suction catheter through the second port and replacing the second cap to seal the second port after removal of the tool, instrument, or suction catheter.
